(19) United States
(12) Patent Application Publication
(10) Pub. No.: US 2002/0132226 A1
(43) Pub. Date: Sep. 19, 2002

(54) Ingestible electronic capsule

(76) Inventors: Vijay Nair, Mesa, AZ (US); Piotr Grodzinski, Chandler, AZ (US); Nada El-Zein, Champaign, IL (US); Herbert Goronkin, Tempe, AZ (US)

Correspondence Address: MOTOROLA, INC., CORPORATE LAW DEPARTMENT - #56-238, 3102 NORTH 56TH STREET, PHOENIX, AZ, 85018, US

(21) Appl. No.: 10/139,868

(22) Filed: May 6, 2002

Related U.S. Application Data
(63) Continuation-in-part of application No. 09/624,807, filed on Jul. 24, 2000, now abandoned.

(51) Int. Cl. C12Q 1/00; G01N 33/53; A61B 5/04

(52) U.S. Cl. 435/4; 600/372

(57) ABSTRACT

An improved and novel ingestible capsule and method for determining medical information from within the alimentary canal of a human or an animal utilizing the ingestible capsule including a non-digestible outer shell that is configured to pass through the alimentary canal. Housed within the non-digestible outer shell is a sensor membrane that is exposed through a portion of the non-digestible outer shell. The sensor membrane is characterized as detecting and identifying predetermined detectable information. Further housed within the non-digestible outer shell are an electronic device that alters its electronic properties in the presence of specific information obtained by the sensor membrane from within the alimentary canal, a bio-sensing circuit that turns on a power source and a low frequency transducer in response to the signal from the electronic device. The low frequency transducer sends a signal of the changed electronic properties to a receiver positioned outside the body.

The present application is related to U.S. patent application Ser. No. 09/624,807 entitled “INGESTIBLE ELECTRONIC CAPSULE” filed Jul. 24, 2000, which is assigned to the current assignee hereof.

### FIELD OF THE INVENTION

The present invention relates to a novel ingestible capsule for use in the field of medicine and method of using the capsule for the accumulation of medical data within the body of animals, and in particular humans.

### BACKGROUND OF THE INVENTION

It is highly desirable to obtain certain medical information and detect certain medical diseases, in particular cancer, without the painful invasive procedures currently used in the medical field. Many of these procedures are unduly stressful and in extreme cases, deter the patient from seeking medical assistance and initial diagnosis. Invasive procedures, or those medical procedures which require the entering of a part of the body, as by incision, scope, etc., are commonly utilized to diagnosis certain diseases and includes procedures such as those utilizing needles, flexible tubes, endoscopic procedures, and surgical procedures.

Many of these diagnostic procedures rely upon the specific procedure or device utilized and the skill of the operator of the device or the one performing the procedure. One such procedure that is typically used today as a common diagnostic tool is colonoscopy for the detection of colorectal cancer (CRC). A colonoscopy generally includes direct visual examination of the colon, ileocecal value, and portions of the terminal ileum by means of a fiberoptic endoscope. A colonoscopy is typically performed by a qualified gastroenterologist. During a colonoscopy the patient is generally awake but sedated. During the procedure a flexible endoscope is inserted in rectum and advanced through the various portions of the lower GI tract. Important anatomic landmarks are identified and surfaces are examined for ulcerations, polyps, hemorrhagic sites, neoplasms, strictures, etc. Dependent upon identified conditions, colorectal cancer, or precancerous conditions of the colon are diagnosed. In many instances, of this invasive procedure, complications arise. The most common complication being perforation of the colon in which diagnosis may be delayed for days until an infection is present. Perforation may be caused by mechanical trauma from the instrument tip, especially if the wall is weakened. Less commonly, perforation may be noninstrumental, secondary to aggressive insufflation with air. However, serious complications from perforation have been reported in routine cases. In addition, hemorrhaging can arise as a complication and many times require repeat colonoscopy to coagulate the bleeding. In a few instances angiography and surgery have been required. A third less common complication is respiratory depression, which is usually due to oversedation in the patient with chronic lung disease.

Other common diagnostic procedures include digital rectal exams, fecal occult blood tests (FOBT) utilizing stool samples, barium enema x-rays, and endoscopic sigmoidoscopy. These procedures are all utilized to diagnose cancerous conditions. During an endoscopic sigmoidoscopy, direct examination of the rectum, sigmoid colon, and proximal portions of the colon (60 cm) is achieved by means of a flexible fiberoptic endoscope. The procedure is generally performed in a physician's office with minimal bowel preparation. The 35 cm scope is more comfortable and less expensive than its larger counterpart, the colonoscopy. Although, the yield of this instrument is somewhat less, with only 40% of malignant or premalignant colonic lesions diagnosed.

All of these procedures are termed invasive procedures and can cause high level of discomfort for the patient. Therefore it is desirable to have a non-invasive procedure that can detect diseases, and/or conditions, such as cancer or the like in their very early stages.

In addition, the medical community has recognized a need for more reliable and less invasive procedures for the detection and thus diagnosis of medical diseases. In recent years “radio pills” have come into being. These pills provide for a means to monitor bodily factors and can either be implanted or ingested and provide for the transmission of information outside of the body. Many of these devices have been quite cumbersome in receiving means, as well as unreliable and generally do not provide for determination of the geographic location of the pill.

Many cancer detection means are known in the medical field, one of such is the use of cancer markers. In order to develop a successful screening procedure for detection of various diseases, including cancer, identification of appropriate and reliable diagnostic markers is essential. There are typically three (3) general categories of such markers: physical, genetic and chemical. One such procedure currently being utilized in the medical field to detect early stages of pre-colon cancer polyp development, is the physical characterization of inner surfaces of the intestine using the endoscopy imaging techniques, such as those previously described with respect to colonoscopy, and flexible sigmoidoscopy. It should be noted that both physical and genetic markers would be difficult to assess using in-vivo detection schemes. Physical markers need to deal with position control, GI tract content interference with the observation, and large amounts of data transmittal. Genetic markers would be difficult to pursue due to the complexity of DNA analysis and detection in a very small volume of the detector. Therefore, chemical detection means are the most logical to pursue for in-vivo mode of detection.

Accordingly, it is an object of the present invention to provide for a device for the detecting and diagnosing of medical conditions utilizing chemical markers.

It is another object of the present invention to provide for a device that is ingestible, such as a capsule, that can transmit diagnostic information to a remote receiver, positioned external to the body, that is reactive upon the sensing of a predetermined factor according to the diagnostic marker utilized.

It is yet another purpose of the present invention to provide for a process of receiving information and diagnosing medical conditions by introducing an ingestible capsule into the body, which is capable of transmitting perceived information based upon detection of a predetermined condition utilizing a chemical marker.

### SUMMARY OF THE INVENTION

These needs and others are substantially met through provision of an ingestible capsule for determining medical information from within the alimentary canal of a human or an animal including a non-digestible outer shell that is configured to pass through the alimentary canal. A sensor membrane is exposed through a portion of the non-digestible outer shell and characterized as detecting a specific condition that is sought to be detected, thereby identifying predetermined detectable information. Housed within the outer shell are an electronic device which includes properties that change in the presence of specific information obtained from the sensor membrane from within the alimentary canal, a bio-sensing circuit that is electronically responsive to the detection of signal from the electronic device, a low frequency transducer that sends a signal of the changed electronic properties outside the body and a miniature battery for powering the transducer.

In addition, disclosed is a method for obtaining diagnostic medical information by ingesting a capsule including a sensor membrane characterized as identifying predetermined detectable information by changing its electrochemical properties, an electronic device that alters its electronic properties due to the chemical behavior changes of the sensor membrane, a low frequency transducer that sends a signal of the changed electronic properties to outside the body, and a miniature battery for powering the transducer. The electronic device is responsive to the changes in the electrical property of the sensor membrane when the membrane interacts with the substance of interest in the tested sample. This change in electrical signal is recognized by the transducer, which submits a signal to a receiver positioned external the body.

### BRIEF DESCRIPTION OF THE DRAWINGS

The foregoing and further and more specific objects and advantages of the instant invention will become readily apparent to those skilled in the art from the following detailed description of a preferred embodiment thereof taken in conjunction with the drawings, in which:

FIG. 1 illustrates a cross-sectional view of an ingestible capsule according to the present invention; and

FIG. 2 illustrates a simplified schematic circuit diagram of the ingestible capsule according to the present invention.

### DETAILED DESCRIPTION OF THE PREFERRED EMBODIMENTS

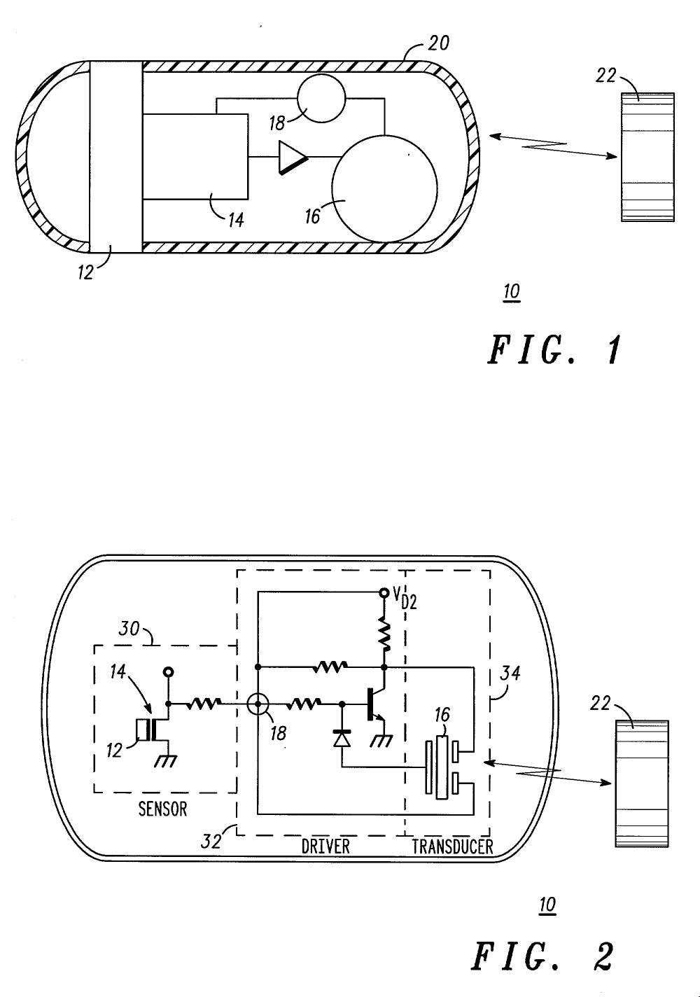

During the course of this description, like numbers are used to identify like elements according to the different figures that illustrate the invention. Accordingly, FIG. 1 illustrates in simplified cross-sectional view an ingestible capsule according to the present invention. More specifically, illustrated in FIG. 1, is an ingestible capsule, designated 10 and the manner in which the components housed with ingestible capsule 10 are interrelated in general. Ingestible capsule 10 typically comprises a sensor membrane 12, an electronic device 14, driver circuit 15, a transducer 16 and a power source 18, such as a miniature battery power source. A dissolvable membrane 11 covers an exposed area of the sensor membrane. Components 12, 14, 16 and 18 are interrelated to provide for the detection of a predetermined factor or condition, such as the presence of an enzyme, antigen, antibody, specific pH level, or the like.

During typical operation, ingestible capsule 10 is swallowed by a “patient” similar to a conventional pill/capsule and propelled through the alimentary canal by natural contractions, called peristalsis. Sensor membrane 12 is coated with chemicals that have specific interaction in the presence of a specific condition, such as a level of enzyme, antigen, antibody, pH, etc. Electronic device 14 is interrelated with the sensor membrane 12 and is characterized as altering its electronic properties in the presence of specific information obtained by sensor membrane 12 and submits an electrical signal that turns “ON” driver circuit 15. Low frequency transducer 16 is then switched on by driver circuit 15 and is characterized as sending a signal of the changed electronic properties to outside the body. This signal of changed electronic properties, meaning the presence of a predetermined factor or condition, is transmitted by transducer 16, in the form of a radio frequency signal, to a receiver 22 that is positioned external the body.

Capsule 10 is fabricated small enough to be easily swallowed by a human or animal. Typically capsule 10 is fabricated less than 11×30 mm, or approximately less than 1″ long, by less than ½″ wide and is fabricated of a sealed, non-digestible outer shell 20, having exposed sensor membrane 12, that is shaped so as to easily pass through the alimentary canal. While it is stated that sensor membrane 12 is exposed to the surrounding environment within the alimentary canal, it should be understood that anticipated by this disclosure is the initial covering of sensor membrane 12 with a dissolvable material. More particularly, it is anticipated that sensor membrane 12 can be initially covered by a dissolvable membrane 11, characterized as dissolving to expose sensor membrane 12 at a specific time/point relative to the alimentary canal. Dissolvable membrane 11 is formed as a protective covering for sensor membrane 12, thereby providing for the protection of sensor membrane 12 from environmental conditions such as stomach acids and degradative enzymes. Dissolvable membrane 11 is fabricated to dissolve at a specific point in time, dependent upon use for ingestible capsule 10. In one specific example, dissolvable membrane 11 is manufactured to dissolve at a point in time, near ingestion of ingestible capsule 10 when test are being run on the esophageal area, etc., such as dissolvable upon contact with saliva of a patient. In a contrasting example, dissolvable membrane 11 is fabricated to dissolve at a point in time in which ingestible capsule 10 would have traveled through the alimentary canal of the patient to the large intestines, when test are being run on the large intestines, such as dissolvable upon contact with a certain pH level found in the large intestines. Once dissolvable membrane 11 covering sensor membrane 12 is dissolved, sensor membrane 12 is exposed to the environment within the alimentary canal of the patient.

Capsule 10 does not include any external wires, fibers, optical bundles or cables, although it is anticipated that capsule 10 can additionally include optical components, etc., to further aid in diagnosing. As previously stated, capsule 10 is propelled by peristalsis, or natural contractions, through the gastrointestinal tract and does not require any pushing force to propel it through the bowel.

The premise for operation of capsule 10 is biosensing. Typically biosensing involves a device that contains biological materials, such as enzymes, cells, antibodies, antigens, or the like, immobilized in conjunction with a transducer which is able to produce an electrical signal when the biological material interacts with the substance of interest in the tested sample. There are several ways to achieve this sensing which can be utilized in capsule 10 of the present invention. More particularly, in capsule 10 of the present invention, sensor membrane 12 is utilized to detect the existence of certain pre-identified condition or material. In one embodiment sensor membrane 12 is formed as a functionalized membrane, such as by including a chemical coating, also known as a chemical marker, that is deposited on the gate of an electronic device 14 such as ion sensitive field effect transistor (ISFET). Sensor membrane 12 is responsive to a specific chemical to be detected, such as that indicative of a cancer precursor. Once the chemical is detected, it will trigger a certain response, such as a voltage change, from the ISFET that can be detected. In particular, the interaction between membrane 12 and the chemical causes the electrical behavior of the FET to change. This change of response of the FET, is monitored to determine the presence of the appropriate chemical, such as glucose, ascorbic, citric acids, or pH levels. Examples of chemical markers which can be utilized to form a functionalized membrane for sensor membrane 12 are found in the following articles: “Glucose, Ascorbic and Citric Acids Detection by two-ISFET Multienzyme Detector”, V. Voltsky, N. Kim, Sensors and Actuators, B 49 (1998), 253-257; “H+ ISFET—Based Biosensor for Determination of Penicillin”, J. Liu, L. Liang, G. Li, R. Han, K. Chen, Biosensors and Bioelectronics, 13 (1998), 1023-1028; and “pH Measurements with an ISFET in the Mouth of Patients with Xerostomia”, L. L. Visch, P. Bergveld, W. Lamprecht, and E. J. Gravenmade, IEEE Transactions on Biomedical Engineering, Vol. 38, No. 4 (1991), 353-356. One example of a chemical marker that can be utilized is the carcinoembryonic antigen (CEA) which is commonly associated with the detection of colon cancer. An antigen is a specific gene product, in most cases a protein, which is foreign to the body and would be recognized by the body immuno-system. The antigen will stimulate the body immuno-system to initiate an immuno-response. One type of the immuno-responses is called humoral immuno response in which the activated B lymphocytes would synthesize, express, and secrete a specific protein product called an antibody which will recognize and bind to the antigen and neutralize it. The inclusion of the CEA with sensor membrane 12 will cause a binding with the antibodies once inside the alimentary canal of a patient in which antibodies receptive to the CEA are found. This binding of the antibodies causes a change in electrical behavior of the ISFET which provides for the ultimate detection of the presence of colon cancer.

In an alternate embodiment of sensor membrane 12, a functionalized electrode, and more particularly, a thin film metal electrode is coated with molecules that are sensitive to the chemical or biological materials that are to be sensed. The thin film metal, such as platinum, gold, or other suitable material can be coated with appropriate chemicals, also known as the chemical marker, to form sensor membrane 12. When sensor membrane 12 is exposed to a specific condition within the alimentary canal of a patient, the molecules present on the electrode bind to the chemical (antibodies or antigens) that is being sensed causing a change in the impedance (i.e. conductivity) of the electrode. As an example, the thin film metal electrode is coated with an antigen specific to an antibody sought to be detected. The antigen binds to the antibodies present in the alimentary canal causing the electrochemical behavior, such as conductivity, to change, and thus indicative of the presence of cancer. This antigen/antibody interaction is specific to a particular type of cancer. Therefore, the change in the electrochemical behavior is a signature of the presence of a particular type of cancerous cell. Electronic device 14 detects this change in conductivity and produces an electronic signal that causes transducer 16 to produce an electronic signal for transmission to outside the body. Examples of this type of sensors are further discussed in the following articles: “Impedimetric Measurements on Polarized Functionalized Platinum Electrodes: Application to Direct Immunosensing”, S. Ameur, H. Maupas, C. Martelet, N. Jaffrezic-Renault, H. Ben Ouada, S. Cosnier, P. Labbe, Materials Science Engineering, C5, (1997), 111-119; and “Sensitive Electrochemical Detection of Antigens Using Gold Electrodes Functionalized with Antibody Moieties”, S. Ameur, C. Martelet, J. M. Chovelon, H. Ben Ouada, N. Jaffrezic-Renault, D. Barbier, Proceedings of the 12^{th} European Conference on Solid State Transducers and the 9^{th} UK Conference on Sensors and their Applications (1998) Vol., 2, 797-800.

The specific embodiments described are the most commonly used electrochemical devices which utilize certain biological/chemical markers for detection purposes. In addition, traditional methods of biological targeting are anticipated for use in ingestible capsule 10 of the present invention, such as, where biological targets are tagged with fluorescent or radioactive molecules and are then imaged through MRI, scopes, or any other optical detection method.

Once the electrochemical characteristics of sensor membrane 12 change in the presence of an identified antigen, antibody, or condition selective to a disease that is sought to be detected, a signal is generated by electronic device 14. This signal turns “ON” driver circuit 15 which triggers the transducer 16 to submit an electronic signal to external receiver 22, either at an ultrasonic frequency or dependent upon a range of detection and sensitivity of the included receiver, from an audio to microwave frequency range. Transducer 16 is described as being a miniature transducer that is fabricated on a ceramic or plastic material. Transducer 16 is fabricated to utilize a very low voltage on the order of 1.5-3.0 volts.

In a preferred embodiment, during operation, the electrical property such as conductivity or potential across electronic device 14 changes. This change of electrical property turns on the transducer 16. Transducer 16 in turn emits a signal as it travels through a region that has activated chemical sensor membrane 12. As ingestible capsule 10 moves away from the region in which chemical sensor membrane 12 is responsive, the transducer is turned off. Accordingly, as the degree of responsiveness increases, as the severity increases. It should be understood that it is anticipated by this disclosure that numerous sensor membranes 12 can be utilized with differing chemical markers, thereby serving as a diagnostic tool for a plurality of conditions, simultaneously. Additionally, a positioning indicator (not shown) can optionally be included for the purpose of determining the exact position of the capsule 10 at any given time in the alimentary canal.

Referring now to FIG. 2, illustrated is a simplified electronic schematic circuit diagram of ingestible capsule 10 of the present invention. Illustrated by dashed lines, is a bio-sensing circuit 30, a driver circuit 32 in electronic communication with bio-sensing circuit 30, and a transducer circuit 34 in electronic communication with driver circuit 32. Bio-sensing circuit 30 includes a sensor membrane 12 and electronic device 14. The sensor membrane is covered by dissolvable membrane 11. Dissolvable membrane 11 dissolves in response to a specific condition thereby exposing sensor membrane 12 through a portion of non-digestible outer shell 20. Sensor membrane 12 is characterized as detecting a specific condition that is sought to be detected, thereby identifying predetermined detectable information. Electronic device 14 is electronically responsive to the detection by sensor membrane 12 of the specific condition and thereby generating a sensing signal. Driver circuit 32, similar to driver circuit 15 of FIG. 1, is electronically responsive to the detection of the sensing signal generated by bio-sensing circuit 30, thereby generating a driving signal. Transducer circuit 34, including transducer 16, is characterized as responsive to the driving signal generated by driver circuit 32, and thereby generating and submitting a radio frequency signal to external receiver 22 positioned outside the body. During operation, in the absence of detection by bio-sensing circuit 30, there is not enough voltage for driver circuit 32 to turn “ON” transducer circuit 34. When biosensing circuit 30 is turned “ON”, due to the reaction of sensor membrane 12 with the detection of an identified material, driver circuit 32 is turned “ON” to produce enough voltage to turn “ON” transducer circuit 34 and thus submit an electronic signal, such as a radio frequency or ultrasonic signal, to receiver 22.

Thus, an ingestible capsule including a small power source, such as a battery, that is connected to a transducer through a bio-sensing circuit is disclosed. When the electrical property such as the conductivity or potential across the electronic device 14 changes, the electrical signal sent out from the bio-sensing circuit 30 turns “ON” driver circuit 32 and thus transducer 16. Transducer 16 then emits an electronic signal to an externally located receiver as it travels through the region in which a predetermined substance of interest has been identified.

What is claimed is:

1. An ingestible capsule for determining medical information in the body of a human or an animal comprising:
a non-digestible outer shell that is configured to pass through an alimentary canal in the body of a human or an animal, the non-digestible outer shell housing within;
a bio-sensing circuit;
a driver circuit in electronic communication with the bio-sensing circuit; and
a transducer circuit in electronic communication with the driver circuit.

2. An ingestible capsule for determining medical information in the body of a human or an animal as claimed in claim 1 wherein the bio-sensing circuit includes a sensor membrane and an electronic device, the sensor membrane covered by a dissolvable membrane, the dissolvable membrane dissolving in response to a specific condition thereby exposing the sensor membrane through a portion of the non-digestible outer shell, the sensor membrane characterized as detecting a specific condition that is sought to be detected, thereby identifying predetermined detectable information, the electronic device, electronically responsive to the detection by the sensor membrane of the specific condition and thereby generating a sensing signal.

3. An ingestible capsule for determining medical information in the body of a human or an animal as claimed in claim 2 wherein the driver circuit is electronically responsive to the detection of the sensing signal generated by the bio-sensing circuit, thereby generating a driving signal.

4. An ingestible capsule for determining medical information in the body of a human or an animal as claimed in claim 3 wherein the transducer circuit includes a low frequency transducer characterized as responsive to the driving signal generated by the driver circuit, and thereby generating and submitting an electronic signal to an external receiver positioned outside the body.

5. An ingestible capsule for determining medical information in the body of a human or an animal as claimed in claim 4 further including a power source for powering the bio-sensing circuit, the driver circuit, and the transducer circuit.

6. An ingestible capsule for determining medical information in the body of a human or an animal comprising:
a non-digestible outer shell that is configured to pass through an alimentary canal in the body of a human or animal, the non-digestible outer shell housing within;
a sensor membrane including an integrally formed chemical marker, the sensor membrane covered by a dissolvable membrane, the dissolvable membrane dissolving in response to a specific condition thereby exposing the sensor membrane through a portion of the non-digestible outer shell, the sensor membrane characterized as sensing a specific condition that is sought to be sensed thereby identifying predetermined detectable information;
an electronic device that is electronically responsive to the sensing by the sensor membrane of the specific condition;
a driver circuit that is electronically responsive to the signal submitted by the electronic device;
a low frequency transducer that sends a signal generated in response to the signal received from the driver circuit to a remote receiver positioned outside the body; and
a power source for supplying power to the electronic device, the driver circuit and the low frequency transducer.

7. An ingestible capsule for determining medical information in the body of a human or an animal as claimed in claim 6 wherein the sensor membrane reacts to a pre-identified condition, characterized as one of a level of an enzyme, an antigen, or an antibody.

8. An ingestible capsule for determining medical information in the body of a human or an animal as claimed in claim 6 wherein the electronic device submits an electrical signal which turns on the driver circuit.

9. An ingestible capsule for determining medical information in the body of a human or an animal as claimed in claim 6 wherein the transducer is a miniature transducer formed on a ceramic or a plastic material.

10. An ingestible capsule for determining medical information in the body of a human or an animal as claimed in claim 9 wherein the transducer emits an ultrasonic signal to the remote receiver positioned outside the body.

11. An ingestible capsule for determining medical information in the body of a human or an animal as claimed in claim 6 wherein the transducer emits an audible signal to the remote receiver positioned outside the body.

12. An ingestible capsule for determining medical information in the body of a human or an animal comprising:
a non-digestible outer shell that is configured to pass through an alimentary canal in the body of a human or an animal, the non-digestible outer shell housing within;
a bio-sensing circuit, including a sensor membrane and an electronic device, the sensor membrane covered by a dissolvable membrane, the dissolvable membrane dissolving in response to a specific condition thereby exposing the sensor membrane through a portion of the non-digestible outer shell, the sensor membrane characterized as detecting a specific condition that is sought to be detected, thereby identifying predetermined detectable information, the electronic device, electronically responsive to the detection by the sensor membrane of the specific condition and thereby generating a sensing signal;
a driver circuit that is electronically responsive to the detection of the sensing signal generated by the bio-sensing circuit, thereby generating a driving signal;
a transducer circuit, including a low frequency transducer characterized as responsive to the driving signal generated by the driver circuit, and thereby generating and submitting an electronic signal to an external receiver positioned outside the body; and
a power source for powering the bio-sensing circuit, the driver circuit, and the transducer circuit.

13. An ingestible capsule for determining medical information in the body of a human or an animal as claimed in claim 12 wherein the sensor membrane includes a chemical marker.

14. An ingestible capsule for determining medical information in the body of a human or an animal as claimed in claim 12 wherein the sensor membrane includes a chemical marker for reacting to a pre-identified condition, evidenced by the presence of one of an enzyme, an antigen, or an antibody.

15. An ingestible capsule for determining medical information in the body of a human or an animal as claimed in claim 12 wherein the power source is a miniature battery.

16. An ingestible capsule for determining medical information in the body of a human or an animal as claimed in claim 12 wherein the transducer is a miniature transducer formed on a ceramic or a plastic material.

17. An ingestible capsule for determining medical information in the body of a human or an animal as claimed in claim 12 wherein the electronic device is one of an ISFET or a functionalized metal electrode.

18. An ingestible capsule for determining medical information in the body of a human or an animal as claimed in claim 12 wherein the transducer emits an audible signal to an externally remote receiver.

19. An ingestible capsule for determining medical information in the body of a human or an animal as claimed in claim 12 wherein the transducer emits an ultrasonic signal to an externally remote receiver.

20. A method for obtaining diagnostic medical information comprising the steps of:
ingesting into the alimentary canal of a human or animal, an electronic capsule including a sensor membrane characterized as identifying predetermined detectable information, an electronic device that alters its electronic properties in the presence of a chemical reaction between the sensor membrane and a specific enzyme, antibody or antigen and generates a sensing signal in response thereto, a driver circuit that turns on and off a power source in response to the sensing signal generated by the electronic device, a low frequency transducer for generating and sending an electronic signal of the changed electronic properties to outside the body, and a miniature battery for powering the driver circuit; and remotely positioning a receiver characterized as receiving the electronic signal from the low frequency transducer.
